(19) United States
(12) Patent Application Publication
(10) Pub. No.: US 2009/0093883 A1
(43) Pub. Date: Apr. 9, 2009

(54) Interspinous implant

(76) Inventor: Mauricio Rodolfo Carrasco, Buenos Aires (AR)

Correspondence Address: ARENT FOX LLP, 1050 CONNECTICUT AVENUE, N.W., SUITE 400, WASHINGTON, DC, 20036, US

(21) Appl. No.: 11/905,982

(22) Filed: Oct. 5, 2007

(51) Int. Cl. A61F 2/44

(52) U.S. Cl. 623/17.16

(57) ABSTRACT

This invention consists in an implant to be placed between the lumbar spinous and lumbar-sacral apophysis in order to keep them separated and enable a normal flexion and extension range. The implant is a cuneiform body which frontal end is rounded and higher than the rear end thus forming an open angle towards the frontal part of the implant. It has two supporting surfaces, opposite to the spinous apophysis and having in its most rear end two sharp edges which are opposite to the spine and located transversally to the longitudinal axis of said faces, and wherein said supporting surfaces are confined by two rhomboidal surfaces which major axis is perpendicular to the longitudinal axis of the implant.

This invention consists in an implant to be placed between the lumbar spinous and lumbar-sacral apophysis in order to keep them separated and enable a normal flexion and extension range.

### BACKGROUND OF THE INVENTION

Several inventions have been disclosed to be inserted between the spinous apophysis. Said inventions have their antecedents in bone grafts placed between the spines more than fifty years ago. They were “H”-shaped and placed so that their ends surrounded the spines and their horizontal part was located between said spines in order to diminish the mobility among the vertebrae and achieve its final fusion.

Likewise, there exist antecedents related to the vertebral fusion which used different bow types, mostly metal bows to be linked to the spinous apophysis so that they become immobilized. The association of both techniques has also been disclosed.

All of said prior art may be found abundantly in the medicine literature.

Afterwards, several inventions having a similar “H” shape were made but aiming to reduce the mobility range among the vertebrae without the fusion thereof and most of said inventions were supplemented with different bows of the spines. The basic action mechanism of said inventions is double since firstly it separates the vertebral bodies and after that, it reduces the relative movement among them.

The U.S. Pat. No. 6,946,000 to Senegas refers to a wedge having two channels in the middle of the implant body to provide said body with flexibility and side walls forming it, which aim consists in opposing an elastic strength to the vertebrae in all of their movements and it is fixed by means of a bow to the neighbouring vertebrae. In this way, Senegas' implant is subject to a permanent deformation stress which may result in the breakage thereof due to the material fatigue. Furthermore, said deformation causes a reduction of the separation desired among the vertebrae. Likewise, there does not exist any angular correction in Senegas' implant according to what is claimed:” . . . the axis of said openings and the axis of said two groves are substantially parallel to each other.” Finally, the design to be adapted to the apophysis does not enable the use of the implant between the lumbar-sacral spinous apophysis.

The invention commercialized under the name X-STOP is a metal oval covered by a plastic material ring having more flexibility than metal, with two side wings, placed between the spines, it resists the stretching movements of the vertebrae due to the strength and flexibility of the material that has been placed thereinbetween. It does not arise from the implant brochure or from the market pieces that said implant introduces any angular correction among the vertebrae.

U.S. Pat. No. 5,645,599 to Samani, is a deformable “U”-shaped sheet having flaps confining the spines and said sheet resists the vertebral movements in the three space plans and the elastic extension and deformation of an elastic curved sheet. As it may be seen from the figures and brochures of the product, one of the implant arms is longer than the other one. As the U arms are projected, the longest arm supports bigger flexion stresses and, consequently, it exhibits a bigger breakage risk due to the material fatigue. Likewise, the flexion reaction stress is also directly supported by the spines. The insertion of said implants in a successive way would cause a very intense stress of the spines with regard to the extension and flexion movements.

The invention disclosed in U.S. Pat. No. 6,626,944 to Jean Taylor is of silicone, and it is deformed and placed between the spines, bowing them, and resists against the flexoextension by the elastic deformation of the silicon. Said document does not mention anything about angular corrections.

In general, these inventions separate the spines leaving them with the same original anatomic relationship or said inventions separate them in such a way that a divergence of the spines is caused backwards, thus causing an inversion of the normal lordosis.

This undesirable effect may be seen in the surgical technique brochure of SFMT EUROPE B.V., wherein there is a reference to the usefulness of the implant and system: “X-STOP, IPD System. A clinical case is shown there and it is shown in a radiograph before and after the insertion of the implant commercially known as “X-Stop”.

Another problem of the interspinous implants is that they cannot be inserted between the fifth lumbar vertebra and the sacrum. This fact relates to the smallness of the spinous apophysis of the sacrum and the lack of the design adaptation of the known implants. That is the case mentioned in the so called WHITE PAPER: “Dynamic lumbar stabilization” The Abbot Spine Walls interespinous implant” (Thieme Medial Publishers) referring to U.S. Pat. No. 6,946,000, which states the need of creating another alternative design for said insertion. The same may be said about the invention disclosed in U.S. Pat. No. 5,645,599, commercially named “Coflex”, wherein in the commercial prospectus the “Paradigm Spine: Coflex, Interspinous implant, Indications and Surgical Technique Prospectus of Coflex” is contraindicated for use in the L5-sacral space due to the technique impossibility.

### BRIEF DESCRIPTION OF THE DRAWINGS

FIG. 1. Front top oblique view of the implant

FIG. 2. Rear top oblique view of the implant

FIG. 3. Top view of the implant

FIG. 4. Lateral view of the implant

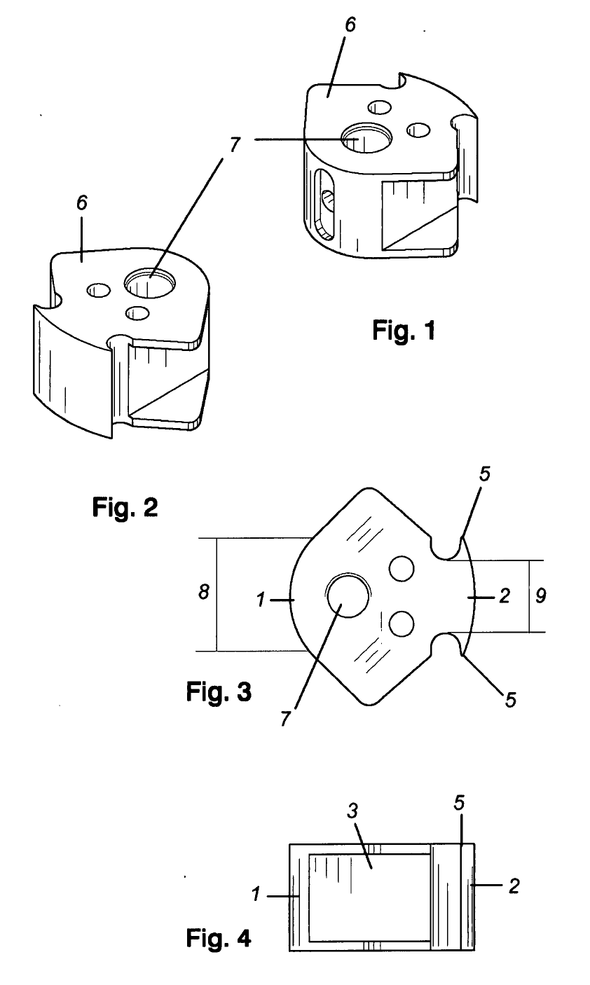

### DESCRIPTION

The invention described herein, due to its wedge profile and the shape and arrangement of the coupling flap of the spine being arranged in an appropriate angle and having a suitable size for the anatomy of the sacrum and the spine, makes the implant adaptation to said area become possible and be kept in its place. Furthermore, it only separates the vertebrae in their front part thus enabling the mobility of the vertebrae between them, with a rotation radius in that fulcrum, which exhibits a different mechanics for the vertebral separation and the movement related to the vertebrae.

In this way, this invention enables to improve the normal anatomic and operation relationship among the vertebrae thus avoiding the reduction effect of the local lordosis of other inventions.

In one if its embodiments, the invention is a cuneiform body which frontal end (1) is rounded and higher than the rear end (2) thus forming an open angle towards the frontal part of the implant (9 to 8).

It has two supporting surfaces, top (3) and bottom (4)surfaces, opposite to the spinous apophysis and having in its most rear end two sharp edges (5) which are opposite to the spines and located transversally to the longitudinal axis of said faces, and wherein said supporting surfaces (4 and 5) are confined by two rhomboidal surfaces (6) which major axis is perpendicular to the longitudinal axis of the implant and with a tunnel (7) which is transversal to the sagital plan of the implant and located behind said major axis of said rhomboidal surfaces.

In one of its embodiments, the implant is a wedge which front end (8) is of 7 mm of height and its rear end (9) is of 3 mm of height. With the same proportions, there exist different wedge sizes for each particular case.

The procedure for the insertion of the invention requires the following steps:

The position of the patient must enable to reduce the normal lordosis to separate the spinous apophysis to a position similar to that achieved with the person being sat down. Precisely, said position is the most appropriate to obtain a relaxation of the structures of the bone continent and of its nervous system content. This fact arises from well-known clinical aspects, in which patients that feel pain when being in a standing position and walking, feel relieved when they sit down or bend forwards. In this way, the recommended surgical position imitates a relaxation physiological position of the nervous system and that is the position in which the separation of the vertebrae will be fixed with the implant. The supraspinous ligament is separated and the interspinous ligament is dried up. The separation achieved among the spinous apophysis is measured and the implant is placed with its higher part forwards until remaining deeply located separating the vertebrae only in their front part. The surgical position of the spinal column is modified below in order to enable the approach of the spines in their rear part and the supraspinous ligament is stitched to the spinous apophysis. In a further preferred embodiment, once the implant had been inserted, bows are passed by the upper and lower spines from the implant and through the tunnel in the rear part of the implant, tautening the bows and fixing them with each other by means of knots or another item that may fix them.

With the implant being placed in this way, the front end (1) of the implant acts as a fulcrum between the spinous apophysis. The flexion and extension movements of the vertebral bodies take place around said fulcrum and the top and bottom supporting surfaces (3 and 4) of said implant serve to limit the movement of the spines during the maximum extension of the spinal column and limit the implant ejection movement.

1. An intervertebral implant to be inserted between the spinous apophysis characterized in that it is a trapezoidal body wherein the major base or front base is slightly rounded, it also has a minor base or rear part and the side walls of the implant or supporting surfaces are opposite, one of them being a top supporting surface and the other one, a bottom supporting surface to support at least one spinous apophysis.

2. The implant of claim 1, characterized in that the bottom and top surfaces form an angle between them which may range between 0° to 15° which is open towards the front part of the implant.

3. The implant of claim 1, characterized in that the length of the implant, said length being the distance between the major base and the minor base, is in the range of 10 mm to 22 mm.

4. The implant of claim 1, characterized in that the front end of the top and bottom surfaces, has in each of them at least a penetrating edge, located transversally to the longitudinal axis of said surfaces.

5. The implant of claim 1, characterized in that the implant includes two side supporting surfaces having a rhomboidal shape, which major axis is perpendicular to the longitudinal axis of the implant.

6. The implant of claim 1, characterized in that it includes at least a tunnel located transversally to the sagital plan of the implant.

7. The implant of claim 1, characterized in that it includes at least a tunnel through which fixing means may be pass and said tunnel is located behind said major axis of said rhomboidal surfaces.

8. The implant of claim 1, characterized in that the fixing means of the implant to the vertebrae or other implants may be elastic in different degrees and materials.

9. The implant of claim 1, characterized in that said fixing means of the implant to the vertebrae or other implants may be made of materials such as metal, plastic, elastic, gums, silicone or the combination thereof.
